(19) United States
(12) Patent Application Publication
(10) Pub. No.: US 2009/0105548 A1
(43) Pub. Date: Apr. 23, 2009

(54) In-Ear Biometrics

(76) Inventor: Gary F. Bart, Miramar, FL (US)

Correspondence Address: HUSCH BLACKWELL SANDERS LLP, 4801 Main Street, Suite 1000, KANSAS CITY, MO, 64112, US

(21) Appl. No.: 11/876,772

(22) Filed: Oct. 23, 2007

(51) Int. Cl. A61B 5/00

(52) U.S. Cl. 600/300

(57) ABSTRACT

There is provided according to an embodiment of the present a biometric tracking Apparatus for use with a digital device, said apparatus comprising: a headphone including a sensor wherein said sensor is configured to produce a data signal that is indicative of one or more biometric parameters; an input configured to receive said data signal; and a storage medium configured to store said data signal.

### CROSS-REFERENCE TO RELATED APPLICATIONS

This application claims priority to U.S. Provisional patent application No. 60/862,379 entitled “In-Ear Biometrics” filed Oct. 20, 2006.

### BACKGROUND OF THE INVENTION

Heart rate monitoring (“HRM”) provides one way to determine and track the intensity of an exercise routine or workout session. An individual can maintain a desired intensity level by monitoring their heart rate during exercise using a heart rate monitor (HRM). Monitoring certain exercise metrics including the intensity and duration of an exercise session can provide an individual with useful information that may be used to establish and monitor fitness goals.

An individual can track their consistency and progress towards reaching their fitness goals by capturing and/or tracking exercise metrics. While exercising it is generally understood that it is desirable to reach and maintain a target workout intensity level. Certain aspects of an exercise session need to be monitored in order for the exerciser to be aware of his current workout intensity so that he may determine if it needs to be adjusted.

Many commonly available HRM devices make use of a sensor and receiver and are available in several configurations. One HRM configuration involves integration of the sensor and receiver into an exercise machine, such as a treadmill or elliptical trainer. An example of this is an HRM that is integrated into cardio-exercise equipment that can be found at many fitness centers. A drawback common to many integrated HRM devices is that these devices are generally limited to monitoring and displaying heart rate information.

Conventional integrated HRM devices do not provide means for a user to easily track or retain exercise heart rate information. In available devices, an individual must manually capture this information by entering it into, for example, a written exercise journal. Even if manually tracked, the user is likely to be capturing only summary heart rate information following a session on the particular exercise machine as opposed to detailed, contemporaneous information about an entire exercise session. Thus, integrated machines do not provide a convenient way for a user to retain/track detailed heart rate information during the course of a session on the machine.

Another popular HRM configuration is a portable, personal HRM unit. Portable units often consist of a watch receiver worn on the wrist of the user and a transmitter that is placed into contact with the skin of the user and generally worn around the chest. In operation, the chest-mounted transmitter picks up the signals from the heart of the wearer, and then sends signals to the watch receiver.

While portable, these HRM devices are not without drawbacks. Among the drawbacks is the expense associated with purchasing a personal HRM. Additionally, it can be inconvenient to use a personal HRM device since this is yet another device to bring and use during an exercise session. Additionally, since personal HRM devices are generally only worn during exercise (if at all), it may be difficult to ascertain a user's resting heart rate and temperature. Thus, use of a conventional personal HRM is often limited because the transmitter and receiver device are only worn during exercise. As a consequence, a large volume of historical biometric data that is not being monitored or captured during non-exercise periods.

Another drawback of known HRM devices is that it is often not possible to store historical data. This may be due to limited internal memory of the receiver device. Further, even when monitored information is stored in a conventional personal HRM device, it is often difficult to access the HRM information. As a result, it is often unduly complicated to extract heart rate data and transfer it to a computer or other external (to the device) storage repository for reporting and analysis. The transfer problem is further exacerbated by use of the proprietary hardware and software interfaces often found in commonly available personal HRM devices.

Given the current popularity and level of interest in fitness, there is no shortage of personal HRM devices. However, despite the easy availability of these devices, relatively few individuals have them or have opted to use them. Thus, there is a need in the art for a device that does not include the drawbacks of HRM devices that are currently available.

### SUMMARY OF THE INVENTION

The foregoing needs are met, to a great extent, by the present invention, wherein in one aspect an apparatus is provided that in some embodiments a biometric sensor is used in conjunction with a digital device, such as a digital media playback device. According to an embodiment of the present invention and method, biometric attributes such as pulse and body temperature can be monitored and stored on such digital device. It will be appreciated that, in addition, heart rate and temperature may also be monitored and measured according to the present invention. An advantage of the present invention is that it substantially solves the problem of users not wanting to use additional equipment for monitoring during a workout.

In one embodiment, biometric attributes are monitored by one or more sensors. According to some embodiments of the present invention, the sensor is a microphone configured to filter out irrelevant/extraneous noise via DSP or other means. At least one of the headphones is capable of sensing one or more biometric attributes. According to another embodiment of the invention, stereo drivers are used and external noise is removed from biometric data by way of a diversity antenna type arrangement.

Certain embodiments of the invention are outlined above in order that the detailed description thereof may be better understood, and in order that the present contributions to the art may be better appreciated. There are, of course, additional embodiments of the invention that will be described below and which will form the subject matter of the claims appended hereto.

In this respect, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention. Though some features of the invention may be claimed in dependency, each feature has merit when used independently.

### BRIEF DESCRIPTION OF THE SEVERAL VIEWS OF THE DRAWING

Further features of the present invention will become apparent to those skilled in the art to which the present invention relates from reading the following description with reference to the accompanying drawings, in which:

FIG. 1 is a perspective view of components used in the environment of the present inventive method and system.

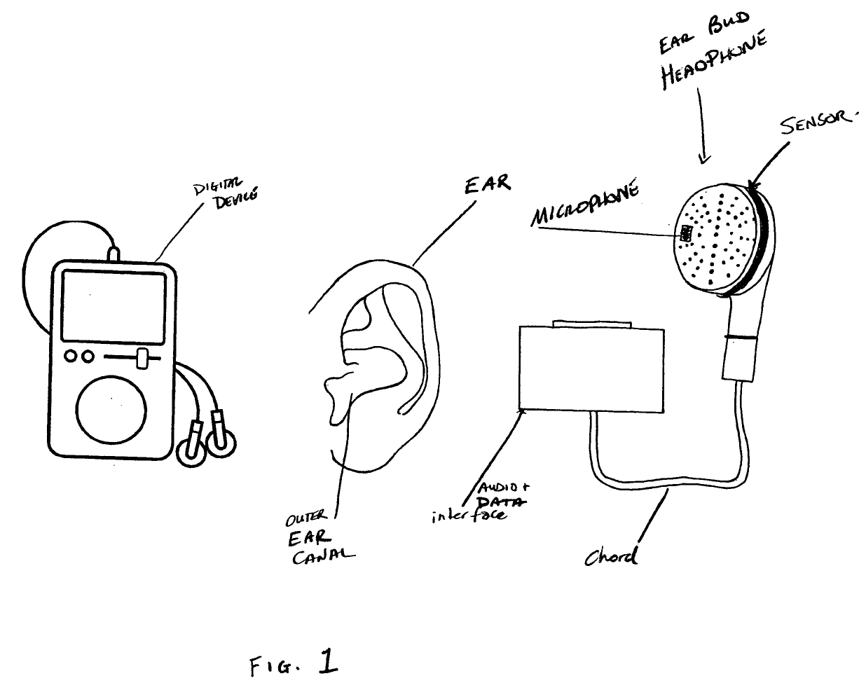

FIG. 2 is a perspective view of an alternate embodiment of components used in the environment of the present inventive method and system.

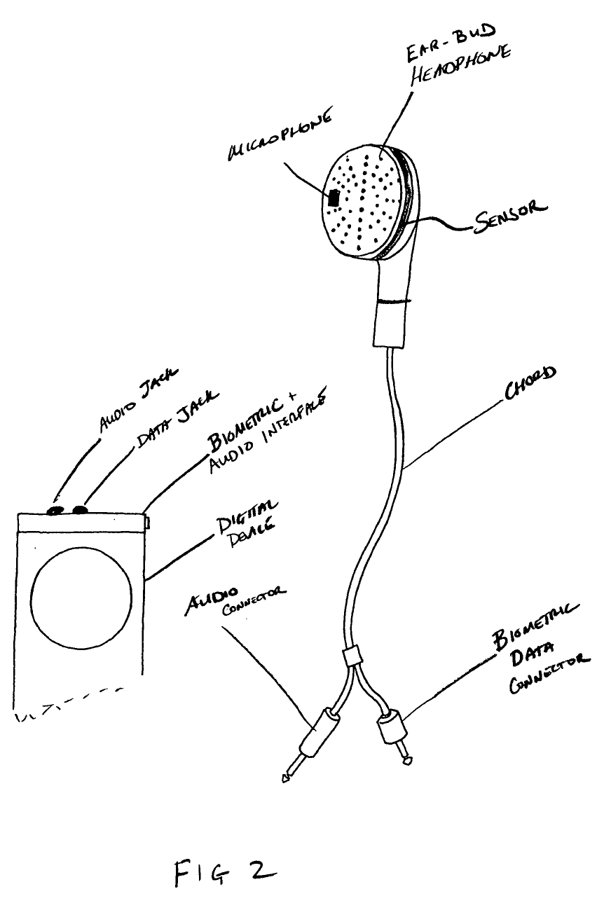

FIG. 3 a perspective view of an alternate embodiment of components used in the environment of the present inventive method and system.

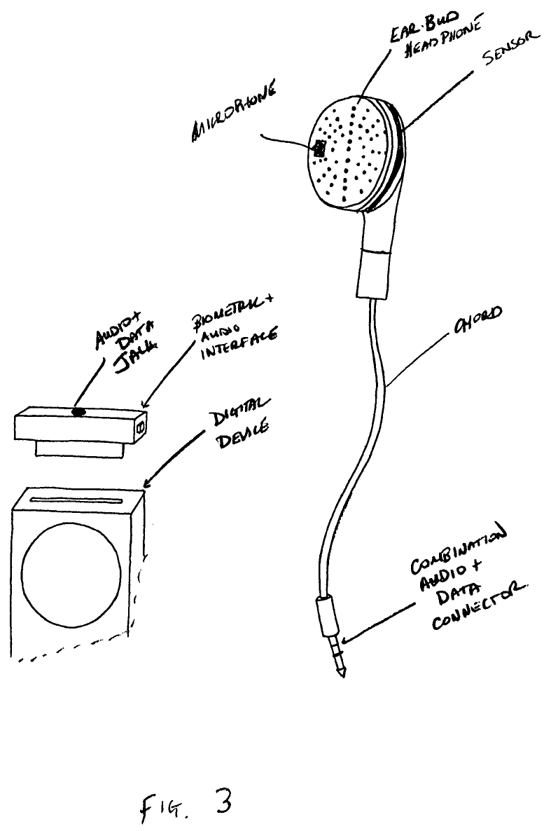

FIG. 4 is a perspective view of an alternate embodiment of components used in the environment of the present inventive method and system.

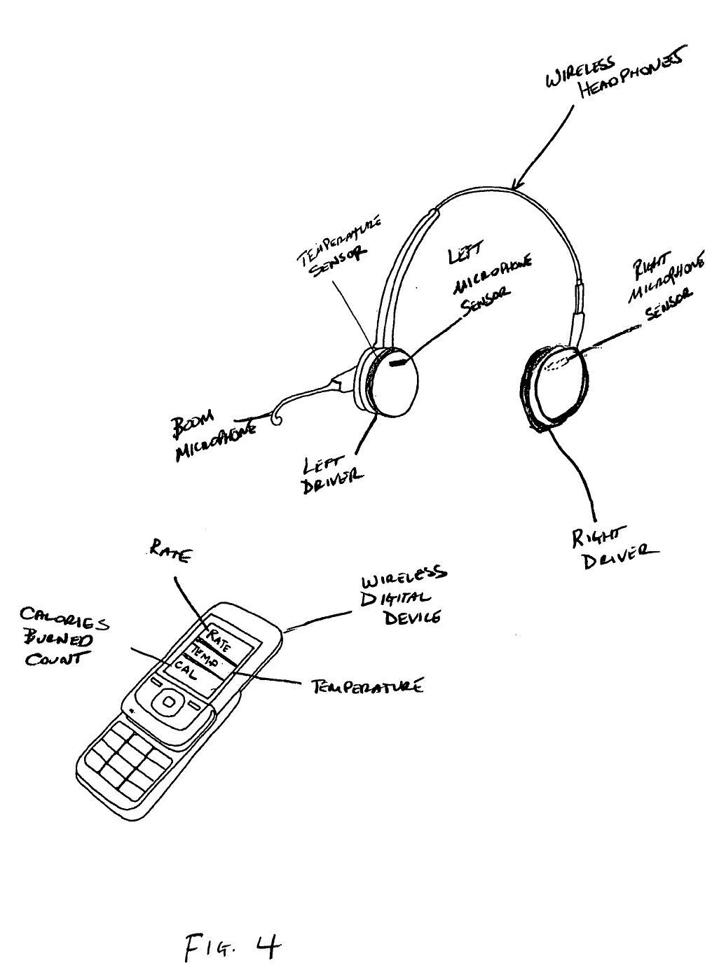

FIG. 5 is a simplified view of a data log used in accordance with an embodiment of the present invention.

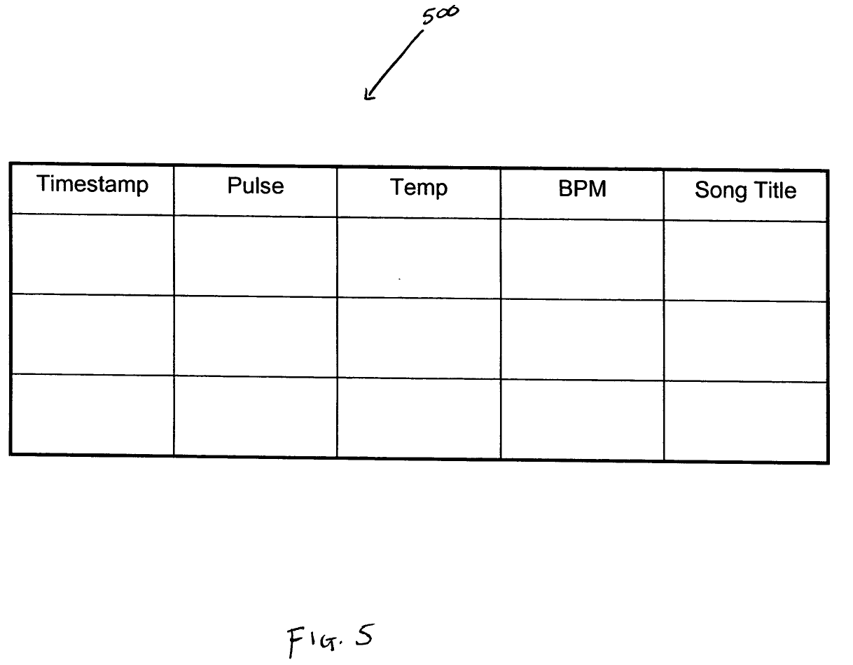

### DETAILED DESCRIPTION OF THE INVENTION

In the present invention, monitored biometric attributes include heart rate and body temperature. It will be appreciated that, in addition, heart rate and temperature may also be monitored and measured according to the present invention. An advantage of the present invention is that it substantially solves the problem of users not wanting to use additional equipment for monitoring during a workout.

In one embodiment, biometric attributes are monitored by one or more sensors. According to some embodiments of the present invention, the sensor is a microphone configured to filter out irrelevant/extraneous noise via DSP or other means. At least one of the headphones is capable of sensing one or more biometric attributes.

In the present invention, monitored biometric attributes include heart rate and body temperature. It will be appreciated that, in addition, heart rate and temperature may also be monitored and measured according to the present invention. An advantage of the present invention is that it substantially solves the problem of users not wanting to use additional equipment for monitoring during a workout.

In one embodiment, biometric attributes are monitored by one or more sensors. According to some embodiments of the present invention, the sensor is a microphone configured to filter out irrelevant/extraneous noise via DSP or other means. At least one of the headphones is capable of sensing one or more biometric attributes.

A number of people exercise while listening to a portable digital media player such as an Apple® iPod® or other device. There are greater numbers of people using a media player while exercising than are using conventional HRM devices that are currently available to consumers.

Digital media players are becoming ubiquitous to exercise and, when used with an biometric sensing earphones according to the present invention, the digital media player becomes a useful component that can be used to keep track of exercise-related data. Current exercise sensing and monitoring tools do not interface with digital media players to keep track of a user's temperature, heart rate, or pulse. It will be appreciated that, as used herein, a digital media player should be broadly construed to include a device that is capable of playing digital media. Examples of digital media players include the Apple® iPod® and mobile phones with media playback functionality.

There are two key biometric parameters: 1) heart rate; and 2) body temperature. One aspect of the present invention includes headphones/earbuds or the like with sensors for temperature on the outer ring and the equivalent of a noise-canceling type sensor microphone for in-ear listening to a heart beat. In another embodiment, the pulse is sensed via skin tissue contact with the earphone by eliminating extraneous vibrations that do not occur at the frequency within that of a normal heartbeat.

According to the teachings of the present invention, the receiver may be connected to one or more of the biometric sensing earphones via a headphone chord. It will be appreciated that the headphone chord may comprise one or more connectors including a standard headphone connector and a second connector to carry the sensor data. Other embodiments may include a single connector that is connected to an adapter (with diplexer/multiplexer-type functionality) in that the adapter splits/extracts the data from the audio media information. According to another embodiment of the invention, the receiver/input adapter includes memory internal to the adapter and serves as a data logger with respect to the sensor information.

While certain embodiments of the present invention feature a receiver connected to the headphone/sensor via a wired connection, one skilled in the art will appreciate that the receiver may also be connected to or communicate with the earphone sensor via a wireless connection. Suitable wireless connections may include Bluetooth®, Zigbee®, 2.4 gigahertz, 5.8 gigahertz, infrared, wireless usb, and other non-wired communication means according to some embodiments of the present invention. In other embodiments of the present invention, the receiver may be integrated into the headphone jack or comparable headphone connector.

According to one embodiment of the present invention, a media player may include a mobile phone with wireless data communication features such as Bluetooth®. In the case of a Bluetooth®-capable phone, one or more of the wireless earphone accessories may include a sensor. In another embodiment, a wireless ear bud headphone includes a sensor that includes a microphone having DSP processing for monitoring the heart beat of the wearer. According to other embodiments of the present invention, a single microphone is used to communicate with callers or interface with the phone via voice commands and monitor the heart beat of the wearer.

An advantage of using wireless technology such as Bluetooth® is that the sensor information may be transmitted to the media player for monitoring via an existing data protocol/profile.

Irrespective of whether or not the sensor communicates with the receiver via a wired or wireless connection, the data signal from the sensor may be transmitted to the receiver in a natively handled format/protocol that is stored in the media player (or in an adapter with data logger) in a readily accessible/parsible format such as a CSV text file, xml or some other extensible data format. Processing duties, reporting, and analysis may be performed on a desktop computer when the media player is docked, connected, and/or synchronized.

According to some embodiments of the present invention, when more than one person makes use of a particular media player, there is an option to select a user profile so that the tracked data is attributed to the person the unit is actually monitoring.

Another advantage of the present invention is that its configuration makes it more likely for biometric information to be monitored and tracked even while the wearer is not exercising. When the biometric sensor of the present invention is in connection with a wireless device with communication functionality, an emergency number can be called to alert a contact that one or more monitored biometric values of the wearer has fallen below a threshold value. For example, an emergency contact number could be automatically called to assist a wearer whose biometric readings indicate that the wearer's heart rate has dropped below a threshold value while the temperature portion of the biometric readings indicate that the earphones are still in use.

Certain embodiments of the invention are outlined above in order that the detailed description thereof may be better understood, and in order that the present contributions to the art may be better appreciated. There are, of course, additional embodiments of the invention that will be described below and which will form the subject matter of the claims appended hereto.

In this respect, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention. Though some features of the invention may be claimed in dependency, each feature has merit when used independently.

One of ordinary skill in the art will appreciate that the processes of the present invention are capable of being distributed in the form of a computer-readable medium of instructions and a variety of other suitable forms and that the present invention applies equally regardless of the particular type of signal-bearing media actually used to carry out the distribution.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

We claim:

1. A biometric tracking apparatus for use with a digital device, said apparatus comprising:
a headphone including a sensor wherein said sensor is configured to produce a data signal that is indicative of one or more biometric parameters;
an input configured to receive said data signal; and
a storage medium configured to store said data signal.

2. The biometric tracking apparatus of claim 1 wherein said digital device is a mobile phone.

3. The biometric tracking apparatus of claim 1 wherein said biometric tracking apparatus features a configurable or selectable polling period.
